(19) United States
(12) Patent Application Publication
(10) Pub. No.: US 2019/0218504 A1
(43) Pub. Date: Jul. 18, 2019

(54) METHODS OF CULTURING AURANTIOCHYTRIUM USING ACETATE AS AN ORGANIC CARBON SOURCE

(71) Applicant: HELIAE DEVELOPMENT LLC, GILBERT, AZ (US)

(72) Inventors: Eneko Ganuza Taberna, Tempe, AZ (US); Charles Sellers, Mesa, AZ (US); April Cobos, Gilbert, AZ (US); Alexander Sitek, Mesa, AZ (US); Jon Hansen, Chandler, AZ (US); Michael LaMont, Chandler, AZ (US)

(21) Appl. No.: 16/325,137

(22) PCT Filed: Sep. 26, 2017
(86) PCT No.: PCT/US17/53432

§ 371 Date: Feb. 12, 2019

Related U.S. Application Data
(60) Provisional application No. 62/448,810, filed on Jan. 20, 2017.
(60) Provisional application No. 62/402,757, filed on Sep. 30, 2016.

(51) Int. Cl. C12N 1/12; C12P 7/64

(52) U.S. Cl. CPC C12N 1/12; C12P 7/6427; C12N 2500/32; C12N 2501/999; C12N 2500/34

(57) ABSTRACT

Methods of growing organisms in the Thraustochytriaceae family using a supply of at least one of acetate and acetic acid as an organic carbon source are described. Embodiments include using acetate or acetic acid in combination with other organic carbon sources and with a pH auxostat system.

### CROSS REFERENCE TO RELATED APPLICATIONS

This application claims the benefit of U.S. Provisional Patent Applications 62/448,810, filed Jan. 20, 2017, entitled METHODS OF CULTURING AURANTIOCHYTRIUM USING ACETATE AS AN ORGANIC CARBON SOURCE and 62/402,757, filed Sep. 30, 2016, entitled METHODS OF CULTURING AURANTIOCHYTRIUM USING ACETATE AS AN ORGANIC CARBON SOURCE, the contents of each being hereby entirely incorporated by reference.

### BACKGROUND

Culturing microalgae in the Thraustochytriaceae family in heterotrophic conditions has traditionally been performed using glycerol or glucose-based organic carbon sources (See Xie, Y., and Wang, G. (2015). Mechanisms of fatty acid synthesis in marine fungus-like protists. Appl. Microbiol. Biotechnol. 99, 8363-8375. doi: 10.1007/s00253-015-6920-7). Use of such organic carbon sources in a culturing method has been shown to result in productive cultures, but requires the culture conditions to remain sterile in order for the productivity to be realized on a consistent basis. Those skilled in the art will recognize that the term “sterile” when applied to a culture of a target microorganism, such as a particular microalgae (e.g., a Thraustochytriaceae family microalgae, such as an Aurantiochytrium) is used in place of the term “axenic”, meaning that only the target microorganism(s) is/are present in detectable quantities. “Sterile” can also mean completely free of detectable living organisms. The proper interpretation of the term will be clear to those skilled in the art based on the context of its use herein and where the term can be both interpreted as referring to a device or composition that is devoid of all detectable living matter (truly sterile) and/or axenic, both embodiments are intended to be provided by the disclosure (e.g., use of a “sterile” bag system disclosed herein means both bag systems that are sterile prior to culture and that are axenic upon inoculation of the target microalgae organism(s)). The necessity for the sterile conditions stems from the fact while many microalgae, including microalgae in the Thraustochytriaceae family, can efficiently utilize glycerol and glucose as an energy source, but so can contaminating organisms such as bacteria, fungi, and yeast. When glycerol or glucose are used as the organic carbon supply in a heterotrophic microalgae culture that is not sterile, the microalgae face substantial competition from contaminating organisms within the culture. Competition with the contaminating organisms may result in the reducing the amount of resources available for the microalgae (e.g., nutrients, gases) or even on physical attacks on the microalgae, either of which can reduce productivity or even the loss of an entire microalgae culture. These direct and indirect adverse effects from contaminating microorganisms on the microalgae culture have dictated that the most reliable way to culture heterotrophic microalgae with traditional organic carbon sources is to use sterile conditions.

Culturing heterotrophic microalgae in sterile conditions comes at a high cost, especially at the commercial level. Depreciation cost typically accounts for 15-30% of the production costs due to the high capex associated with sterile fermentation technology. Moreover, large microalgae cultures comprise a large number of opportunities for the sterility conditions to be compromised due to the number of inputs in a culture (e.g., media, organic carbon, gases), the scale up process that involves transfer between multiple bioreactor systems, the number of steps in a sterilization protocol for equipment that is of an industrial scale size, and the length of the culturing process (e.g., days or weeks). These potential infiltrations of contaminating organisms, such as bacteria, may occur whether the culture is in a closed or open bioreactor, although the risk is correspondingly higher in an open culture. Accordingly, culturing microalgae heterotrophically in low cost open bioreactor systems faces a heightened level of contamination risk and challenges for reliable production.

### SUMMARY

Methods of growing organisms in the Thraustochytriaceae family using a supply of at least one of acetate and acetic acid as an organic carbon source are described. Embodiments include using acetate or acetic acid in combination with other organic carbon sources and with a pH auxostat system. In one non-limiting embodiment, a method of growing Aurantiochytrium using acetate as the sole organic carbon source or in combination with other organic carbon sources in axenic or non-axenic conditions. A non-limiting embodiment describing a method of scaling up a culture of Aurantiochytrium for a final stage of production in an open outdoor bioreactor is also described.

### BRIEF DESCRIPTION OF THE FIGURE

FIG. 1 shows a graph of cell dry weight for a culture of Aurantiochytrium reflecting an exemplary aspect of the invention.

### DETAILED DESCRIPTION

The inventors have developed a method of increasing reliable production of heterotrophic microalgae, in closed or open bioreactor systems operating in axenic or non-axenic conditions. The term “microalgae” refers to any microorganisms classified as microalgae, cyanobacteria, diatoms, dinoflagellates, or other similar single cell microorganisms, whether freshwater or marine, capable of growth in phototrophic, mixotrophic, or heterotrophic culture conditions. In some non-limiting embodiments, the described methods may be used with microalgae in the Thraustochytriaceae family such as Aurantiochytrium. To increase the reliability of heterotrophic microalgae production and enable culturing in low cost open outdoor bioreactor system, the inventors developed a culturing method in which at least one of acetate and acetic acid is supplied as an organic carbon source while also contributing to the creation of an environment of growth restrictive culture conditions that favor the microalgae over the contaminating organisms, such as bacteria.

Taxonomic classification has been in flux for microalgae in the Thraustochytriaceae family, particularly the genus Schizochytrium. Some microalgae previously classified as Schizochytrium have been reclassified as Aurantiochytrium, Thraustochytrium, or Oblongichytrium. See Yokoyama et al. Taxonomic rearrangement of the genus Schizochytrium sensu lato based on morphology, chemotaxonomic characteristics, and 18S rRNA gene phylogeny (Thrausochytriaceae, Labyrinthulomycetes): emendation for Schizochytrium and erection of Aurantiochytrium and Oblongichytrium gen. nov. Mycoscience (2007) 48:199-211. Those of skill in the art will recognize that Thraustochytrids such as Schizochytrium, Aurantiochytrium, Thraustochytrium, and Oblongichytrium appear closely related in many taxonomic classification trees for microalgae, and strains and species may be re-classified from time to time. Thus for references throughout the instant specification for Aurantiochytrium, it is recognized that microalgae strains in related taxonomic classifications with similar characteristics to Aurantiochytrium would reasonably be expected to produce similar results.

While the developed methods comprising a supply of at least one of acetate and acetic acid have utility in non-axenic conditions found in open outdoor bioreactor systems, the methods are also useful for axenic cultures and sterile bioreactor systems. The utility of the inventive method is supported by the surprising results in the Examples in which the inventors demonstrated that first Aurantiochytrium is capable of utilizing acetate or acetic acid as an organic carbon source, and second Aurantiochytrium is capable of proliferating in an environment where a residual acetate concentration creates an environment that favors Aurantiochytrium over other heterotrophic contaminates (i.e., an environment of acetate toxicity). Using the inventive method, the inventors where able to successfully culture Aurantiochytrium utilizing acetate or acetic acid as an organic carbon source for biomass and lipid production, in axenic and non-axenic conditions, which provides the flexibility of choosing from a variety of bioreactor system and culture conditions for a cost effective commercial process.

The term “pH auxostat” refers to the microbial cultivation technique that couples the addition of fresh medium (e.g., medium containing organic carbon or acetic acid) to pH control. As the pH drifts from a given set point, fresh medium is added to bring the pH back to the set point. The rate of pH change is often an excellent indication of growth and meets the requirements as a growth-dependent parameter. The feed will keep the residual nutrient concentration in balance with the buffering capacity of the medium. The pH set point may be changed depending on the microorganisms present in the culture at the time. The microorganisms present may be driven by the location and season where the bioreactor is operated and how close the cultures are positioned to other contamination sources (e.g., other farms, agriculture, ocean, lake, river, waste water). The rate of medium addition is determined by the substrate consumption rate of the microorganism and the buffering capacity of the media. The pH auxostat controls nutrient concentration indirectly (i.e., based on pH) and therefore the buffering capacity of the media may have an impact on the residual acetate and ultimately in the toxicity of the media. The pH level represents the summation of the production of different ionic species and ion release during carbon and nutrient uptake.

In some embodiments, the inventive method utilizes a pH auxostat to provide at least one of supply acetic acid to the microalgae culture as a source of organic carbon, a method of maintaining the culture pH in a desired range, and a method of maintaining the residual acetate concentration (i.e., acetate toxicity conditions) in a desired range. In some embodiments, the pH auxostat system may comprise a solenoid valve, a peristaltic pump, a pH probe and a pH controller. In some embodiments, the pH auxostat system may comprise a drip application device controlled by a needle valve, a metering pump or a peristaltic pump, and a pH controller. The pH controller may be set at a threshold level (i.e., set point) and activate the auxostat system to supply acetic acid to the culture when the measured pH level is above the set threshold level. The frequency of pH measurements, administration of acetic acid by the auxostat system, and mixing of the culture are controlled in combination to keep the pH value substantially constant. In some embodiments, the acetic acid feed may be diluted in water to a concentration below 100% and as low as 0.5%, with a preferable concentration between 15% and 50%. In other embodiments, the acetic acid may be at concentrations below 10% in order to continuously dilute the culture of microorganisms. In other embodiments, the acetic acid may be mixed together with other nutrient media, acids, or organic carbon sources.

The creation and control of a residual acetate concentration in the culture medium is not inherent in a pH auxostat system. The toxicity of the environment is governed by a variety of factors, such as but not limited to, the total concentration of acetate in the culture and the pH of the culture; and thus the residual acetate concentration forming the toxicity condition is controlled by the initial concentration of acetate and the supply of acetic acid through the pH auxostat. In some embodiments, acetate or acetic acid may be supplied as a batch.

While some microalgae are known to use acetate or acetic acid as a carbon source, the inventors determined that too much acetate can also be toxic to microalgae and thus acetate tolerance limits may vary among microalgae. The higher tolerance limit of microalgae to acetate and undissociated acetic acid, as compared to bacteria, may be attributable to microalgae having differentiated organelles and a nucleus. However, the indiscriminate use of acetic acid in a microalgae culture contaminated with bacteria may negatively affect the bacteria, but may also negatively affect the microalgae if the residual acetate concentration formed in the culture is above the tolerance limit of the microalgae.

Therefore, the residual acetate level could increase or decrease as a function of growth of the microorganisms. The most common situation is an acetate decrease caused by organic acid production and ammonium uptake. However, for microorganisms growing on nitrates, protein or amino acid-rich media, the residual acetate level will rise with growth because the consumption of those nutrients.

### Flask Stage

In some embodiments, the microalgae in the Thraustochytriaceae family may be cultured with at least one of acetate and acetic acid in flasks in an aqueous culture medium before the culture is scaled up to a larger volume and bioreactor. In some embodiments, a flask culture may comprise a volume in the range of 0.1 to 1 liters, a culture temperature in the range of 20° C. to 40° C., a culture pH in the range of 4-9, and an initial inoculation density of 0.1-1.0 g/L. In some embodiments, the flask culture may achieve a culture density of up to 100 g/L.

In some embodiments, the flask culture may be cultured at a temperature in the range of 20-22° C. In some embodiments, the flask culture may be cultured at a temperature in the range of 22-24° C. In some embodiments, the flask culture may be cultured at a temperature in the range of 24-26° C. In some embodiments, the flask culture may be cultured at a temperature in the range of 26-28° C. In some embodiments, the flask culture may be cultured at a temperature in the range of 28-30° C. In some embodiments, the flask culture may be cultured at a temperature in the range of 30-32° C. In some embodiments, the flask culture may be cultured at a temperature in the range of 32-35° C. In some embodiments, the flask culture may be cultured at a temperature in the range of 35-38° C. In some embodiments, the flask culture may be cultured at a temperature in the range of 38-40° C.

In some embodiments, the flask culture may be cultured at a pH in the range of 4-5. In some embodiments, the flask culture may be cultured at a pH in the range of 5-6. In some embodiments, the flask culture may be cultured at a pH in the range of 6-7. In some embodiments, the flask culture may be cultured at a pH in the range of 7-8. In some embodiments, the flask culture may be cultured at a pH in the range of 8-9.

In some embodiments, the flask culture may be inoculated at a density in the range of 0.1-0.2 g/L. In some embodiments, the flask culture may be inoculated at a density in the range of 0.2-0.3 g/L. In some embodiments, the flask culture may be inoculated at a density in the range of 0.3-0.4 g/L. In some embodiments, the flask culture may be inoculated at a density in the range of 0.4-0.5 g/L. In some embodiments, the flask culture may be inoculated at a density in the range of 0.5-0.6 g/L. In some embodiments, the flask culture may be inoculated at a density in the range of 0.6-0.7 g/L. In some embodiments, the flask culture may be inoculated at a density in the range of 0.7-0.8 g/L. In some embodiments, the flask culture may be inoculated at a density in the range of 0.8-0.9 g/L. In some embodiments, the flask culture may be inoculated at a density in the range of 0.9-1.0 g/L. In some embodiments, the flask culture may be inoculated at a density of up to 5 g/L. In some embodiments, the flask culture may be inoculated at a density of up to 10 g/L. In some embodiments, the flask culture may be inoculated at a density of up to 15 g/L. In some embodiments, the flask culture may be inoculated at a density of up to 20 g/L. In some embodiments, it may be preferred to inoculate a culture at a density of less than 10 g/L because it requires less biomass to start the process.

In some embodiments, the flask culture may achieve a culture density in the range of 1-10 g/L. In some embodiments, the flask culture may achieve a culture density in the range of 10-20 g/L. In some embodiments, the flask culture may achieve a culture density in the range of 20-30 g/L. In some embodiments, the flask culture may achieve a culture density in the range of 30-40 g/L. In some embodiments, the flask culture may achieve a culture density in the range of 40-50 g/L. In some embodiments, the flask culture may achieve a culture density in the range of 50-60 g/L. In some embodiments, the flask culture may achieve a culture density in the range of 60-70 g/L. In some embodiments, the flask culture may achieve a culture density in the range of 70-80 g/L. In some embodiments, the flask culture may achieve a culture density in the range of 80-90 g/L. In some embodiments, the flask culture may achieve a culture density in the range of 90-100 g/L.

In some embodiments, the flask culture may achieve a culture density up to 150 g/L. In some embodiments, the flask culture may achieve a culture density up to 200 g/L. In some embodiments, the flask culture may achieve a culture density up to 250 g/L. In some embodiments, the flask culture may achieve a culture density up to 300 g/L. In some embodiments, the flask culture may achieve a culture density up to 400 g/L. In some embodiments, the flask culture may achieve a culture density up to 500 g/L.

### Bag Bioreactor Stage

In some embodiments, the microalgae in the Thraustochytriaceae family may be cultured with at least one of acetate and acetic acid in a bag bioreactor in an aqueous culture medium before the culture is scaled up to a larger volume and bioreactor. In some embodiments, bag bioreactor culture may comprise a volume in the range of 50 to 1,000 liters, a culture temperature in the range of 20° C. to 40° C., a culture pH in the range of 4-9, and an initial inoculation density of 0.1-1.0 g/L. In some embodiments, the bag bioreactor culture may achieve a culture density of up to 100 g/L. In some embodiments, the bag bioreactor culture may achieve a biomass weight yield per gram of organic carbon of up to 0.55. In some embodiments, the bag bioreactor culture may achieve an Omega-3 fatty acid weight yield per gram of organic carbon of up to 0.30. In some embodiments, the bag bioreactor culture may achieve a lipid productivity rate of up to 1.0 g/L/hr.

In some embodiment, the methods of the invention are characterized by increasing the biomass concentration in the culture by any detectable amount. In other aspects, the biomass concentration is increased by at least 10% over the starting biomass. In still further facets, the biomass concentration is increased by at least 50%, such as at least 100% (i.e., doubled or 2×/two-fold) over the starting culture. The biomass concentration can be increased by methods of the invention at least 3× (increased more than 200% or 200%+), at least 4× (increased 300%+), 5× (increased 400%+), at least 6× (increased 500%+), at least 1 Ox (increased 900%+), at least 12× (increased 1100%+), at least 15× (increased 1400%+), at least 20× (increased 1900%/+), at least 25× (increased 2400%+), or even at least 30× (increased 2900%+). In some aspects the increase in concentration of biomass (typically measured in g/L) obtained by practice of methods of the invention is in the range of 500%-4000%, such as 500%-3750%, for example 1000/%-3750%, 2000%-3750%, 1000%-4000%, 2000%-4000%, 2500/%-4000%, 2500%-3750%, 3000%-4000%, or 3000%-3750%.

In some embodiments, the bag bioreactor culture may be cultured at a temperature in the range of 20-22° C. In some embodiments, the bag bioreactor culture may be cultured at a temperature in the range of 22-24° C. In some embodiments, the bag bioreactor culture may be cultured at a temperature in the range of 24-26° C. In some embodiments, the bag bioreactor culture may be cultured at a temperature in the range of 26-28° C. In some embodiments, the bag bioreactor culture may be cultured at a temperature in the range of 28-30° C. In some embodiments, the bag bioreactor culture may be cultured at a temperature in the range of 30-32° C. In some embodiments, the bag bioreactor culture may be cultured at a temperature in the range of 32-35° C. In some embodiments, the bag bioreactor culture may be cultured at a temperature in the range of 35-38° C. In some embodiments, the bag bioreactor culture may be cultured at a temperature in the range of 38-40° C.

In some embodiments, the bag bioreactor culture may be cultured at a pH in the range of 4-5. In some embodiments, the bag bioreactor culture may be cultured at a pH in the range of 5-6. In some embodiments, the bag bioreactor culture may be cultured at a pH in the range of 6-7. In some embodiments, the bag bioreactor culture may be cultured at a pH in the range of 7-8. In some embodiments, the bag bioreactor culture may be cultured at a pH in the range of 8-9.

In some embodiments, the bag bioreactor culture may be inoculated at a density in the range of 0.1-0.2 g/L. In some embodiments, the bag bioreactor culture may be inoculated at a density in the range of 0.2-0.3 g/L. In some embodiments, the bag bioreactor culture may be inoculated at a density in the range of 0.3-0.4 g/L. In some embodiments, the bag bioreactor culture may be inoculated at a density in the range of 0.4-0.5 g/L. In some embodiments, the bag bioreactor culture may be inoculated at a density in the range of 0.5-0.6 g/L. In some embodiments, the bag bioreactor culture may be inoculated at a density in the range of 0.6-0.7 g/L. In some embodiments, the bag bioreactor culture may be inoculated at a density in the range of 0.7-0.8 g/L. In some embodiments, the bag bioreactor culture may be inoculated at a density in the range of 0.8-0.9 g/L. In some embodiments, the bag bioreactor culture may be inoculated at a density in the range of 0.9-1.0 g/L. In some embodiments, the bag bioreactor culture may be inoculated at a density of up to 5 g/L. In some embodiments, the bag bioreactor culture may be inoculated at a density of up to 10 g/L. In some embodiments, the bag bioreactor culture may be inoculated at a density of up to 15 g/L. In some embodiments, the bag bioreactor culture may be inoculated at a density of up to 20 g/L. In some embodiments, it may be preferred to inoculate a culture at a density of less than 10 g/L because it requires less biomass to start the process.

In some embodiments, the bag bioreactor culture may achieve a culture density in the range of 1-10 g/L. In some embodiments, the bag bioreactor culture may achieve a culture density in the range of 10-20 g/L. In some embodiments, the bag bioreactor culture may achieve a culture density in the range of 20-30 g/L. In some embodiments, the bag bioreactor culture may achieve a culture density in the range of 30-40 g/L. In some embodiments, the bag bioreactor culture may achieve a culture density in the range of 40-50 g/L. In some embodiments, the bag bioreactor culture may achieve a culture density in the range of 50-60 g/L. In some embodiments, the bag bioreactor culture may achieve a culture density in the range of 60-70 g/L. In some embodiments, the bag bioreactor culture may achieve a culture density in the range of 70-80 g/L. In some embodiments, the bag bioreactor culture may achieve a culture density in the range of 80-90 g/L. In some embodiments, the bag bioreactor culture may achieve a culture density in the range of 90-100) g/L.

In some embodiments, the bag bioreactor culture may achieve a culture density a culture density up to 150 g/L. In some embodiments, the bag bioreactor culture may achieve a culture density a culture density up to 200 g/L. In some embodiments, the bag bioreactor culture may achieve a culture density a culture density up to 250 g/L. In some embodiments, the bag bioreactor culture may achieve a culture density a culture density up to 300 g/L. In some embodiments, the bag bioreactor culture may achieve a culture density a culture density up to 400 g/L. In some embodiments, the bag bioreactor culture may achieve a culture density a culture density up to 500 g/L.

In some embodiments, the bag bioreactor culture may achieve a biomass weight yield per gram of organic carbon in the range of 0.1-0.2. In some embodiments, the bag bioreactor culture may achieve a biomass weight yield per gram of organic carbon in the range of 0.2-0.3.

In some embodiments, the bag bioreactor culture may achieve a biomass weight yield per gram of organic carbon in the range of 0.3-0.4. In some embodiments, the bag bioreactor culture may achieve a biomass weight yield per gram of organic carbon in the range of 0.4-0.5. In some embodiments, the bag bioreactor culture may achieve a biomass weight yield per gram of organic carbon in the range of 0.5-0.55.

In some embodiments, the bag bioreactor culture may achieve an Omega-3 fatty acid weight yield per gram of organic carbon in the range of 0.10-0.15. In some embodiments, the bag bioreactor culture may achieve an Omega-3 fatty acid weight yield per gram of organic carbon in the range of 0.15-0.20. In some embodiments, the bag bioreactor culture may achieve an Omega-3 fatty acid weight yield per gram of organic carbon in the range of 0.20-0.25. In some embodiments, the bag bioreactor culture may achieve an Omega-3 fatty acid weight yield per gram of organic carbon in the range of 0.25-0.30.

In some embodiments, the bag bioreactor culture may achieve a lipid productivity rate in the range of 0.1-0.2 g/L. In some embodiments, the bag bioreactor culture may achieve a lipid productivity rate in the range of 0.2-0.3 g/L. In some embodiments, the bag bioreactor culture may achieve a lipid productivity rate in the range of 0.3-0.4 g/L. In some embodiments, the bag bioreactor culture may achieve a lipid productivity rate in the range of 0.4-0.5 g/L. In some embodiments, the bag bioreactor culture may achieve a lipid productivity rate in the range of 0.5-0.6 g/L. In some embodiments, the bag bioreactor culture may achieve a lipid productivity rate in the range of 0.6-0.7 g/L. In some embodiments, the bag bioreactor culture may achieve a lipid productivity rate in the range of 0.7-0.8 g/L. In some embodiments, the bag bioreactor culture may achieve a lipid productivity rate in the range of 0.8-0.9 g/L. In some embodiments, the bag bioreactor culture may achieve a lipid productivity rate in the range of 0.9-1.0 g/L.

### Open Bioreactor Stage

In some embodiments, the microalgae in the Thraustochytriaceae family may be cultured with at least one of acetate and acetic acid in an open bioreactor in an aqueous culture medium. In some embodiments, the open bioreactor may be disposed outdoors. In some embodiments, open bioreactor culture may comprise a volume of up to 500,000 liters, a culture temperature in the range of 20° C. to 40° C., a culture pH in the range of 4-9, and an initial inoculation density of 0.1-10 g/L. In some embodiments, the open bioreactor culture may achieve a culture density of up to 200 g/L. In some embodiments, the open bioreactor culture may achieve a biomass weight yield per gram of organic carbon of up to 0.55. In some embodiments, the open bioreactor culture may achieve an Omega-3 fatty acid weight yield per gram of organic carbon of up to 0.30. In some embodiments, the open bioreactor culture may achieve a lipid productivity rate of up to 1.0 g/L/hr. In some embodiments, the open bioreactor culture may achieve an average biomass productivity rate of up to 60 g/L/day.

In some embodiments, the open bioreactor culture may be cultured at a temperature in the range of 20-22° C. In some embodiments, the open bioreactor culture may be cultured at a temperature in the range of 22-24° C. In some embodiments, the open bioreactor culture may be cultured at a temperature in the range of 24-26° C. In some embodiments, the open bioreactor culture may be cultured at a temperature in the range of 26-28° C. In some embodiments, the open bioreactor culture may be cultured at a temperature in the range of 28-30° C. In some embodiments, the open bioreactor culture may be cultured at a temperature in the range of 30-32° C. In some embodiments, the open bioreactor culture may be cultured at a temperature in the range of 32-35° C. In some embodiments, the open bioreactor culture may be cultured at a temperature in the range of 35-38° C. In some embodiments, the open bioreactor culture may be cultured at a temperature in the range of 38-40° C.

In some embodiments, the open bioreactor culture may be cultured at a pH in the range of 4-5. In some embodiments, the open bioreactor culture may be cultured at a pH in the range of 5-6. In some embodiments, the open bioreactor culture may be cultured at a pH in the range of 6-7. In some embodiments, the open bioreactor culture may be cultured at a pH in the range of 7-8. In some embodiments, the open bioreactor culture may be cultured at a pH in the range of 8-9.

In some embodiments, the open bioreactor culture may be inoculated at a density in the range of 0.1-0.2 g/L. In some embodiments, the open bioreactor culture may be inoculated at a density in the range of 0.2-0.3 g/L. In some embodiments, the open bioreactor culture may be inoculated at a density in the range of 0.3-0.4 g/L. In some embodiments, the open bioreactor culture may be inoculated at a density in the range of 0.4-0.5 g/L. In some embodiments, the open bioreactor culture may be inoculated at a density in the range of 0.5-0.6 g/L. In some embodiments, the open bioreactor culture may be inoculated at a density in the range of 0.6-0.7 g/L. In some embodiments, the open bioreactor culture may be inoculated at a density in the range of 0.7-0.8 g/L. In some embodiments, the open bioreactor culture may be inoculated at a density in the range of 0.8-0.9 g/L. In some embodiments, the open bioreactor culture may be inoculated at a density in the range of 0.9-1.0 g/L. In some embodiments, the open bioreactor culture may be inoculated at a density of up to 5 g/L. In some embodiments, the open bioreactor culture may be inoculated at a density of up to 10 g/L. In some embodiments, the open bioreactor culture may be inoculated at a density of up to 15 g/L. In some embodiments, the open bioreactor culture may be inoculated at a density of up to 20 g/L. In some embodiments, it may be preferred to inoculate a culture at a density of less than 10 g/L because it requires less biomass to start the process.

In some embodiments, the open bioreactor culture may achieve a culture density in the range of 1-10 g/L. In some embodiments, the open bioreactor culture may achieve a culture density in the range of 10-20 g/L. In some embodiments, the open bioreactor culture may achieve a culture density in the range of 20-30 g/L. In some embodiments, the open bioreactor culture may achieve a culture density in the range of 30-40 g/L. In some embodiments, the open bioreactor culture may achieve a culture density in the range of 40-50 g/L. In some embodiments, the open bioreactor culture may achieve a culture density in the range of 50-60 g/L. In some embodiments, the open bioreactor culture may achieve a culture density in the range of 60-70 g/L. In some embodiments, the open bioreactor culture may achieve a culture density in the range of 70-80 g/L. In some embodiments, the open bioreactor culture may achieve a culture density in the range of 80-90 g/L. In some embodiments, the open bioreactor culture may achieve a culture density in the range of 90-100 g/L.

In some embodiments, the open bioreactor culture may achieve a culture density a culture density up to 150 g/L. In some embodiments, the open bioreactor culture may achieve a culture density a culture density up to 200 g/L. In some embodiments, the open bioreactor culture may achieve a culture density a culture density up to 250 g/L. In some embodiments, the open bioreactor culture may achieve a culture density a culture density up to 300 g/L. In some embodiments, the open bioreactor culture may achieve a culture density a culture density up to 400 g/L. In some embodiments, the open bioreactor culture may achieve a culture density a culture density up to 500 g/L.

In some embodiments, the open bioreactor culture may achieve a biomass weight yield per gram of organic carbon in the range of 0.1-0.2. In some embodiments, the open bioreactor culture may achieve a biomass weight yield per gram of organic carbon in the range of 0.2-0.3 g. In some embodiments, the open bioreactor culture may achieve a biomass weight yield per gram of organic carbon in the range of 0.3-0.4. In some embodiments, the open bioreactor culture may achieve a biomass weight yield per gram of organic carbon in the range of 0.4-0.5.

In some embodiments, the open bioreactor culture may achieve a biomass weight yield per gram of organic carbon in the range of 0.5-0.55.

In some embodiments, the open bioreactor culture may achieve an Omega-3 fatty acid weight yield per gram of organic carbon in the range of 0.01-0.05. In some embodiments, the open bioreactor culture may achieve an Omega-3 fatty acid weight yield per gram of organic carbon in the range of 0.05-0.10. In some embodiments, the open bioreactor culture may achieve an Omega-3 fatty acid weight yield per gram of organic carbon in the range of 0.10-0.15 In some embodiments, the open bioreactor culture may achieve an Omega-3 fatty acid weight yield per gram of organic carbon in the range 0.15-0.20. In some embodiments, the open bioreactor culture may achieve an Omega-3 fatty acid weight yield per gram of organic carbon in the range of 0.20-0.25. In some embodiments, the open bioreactor culture may achieve an Omega-3 fatty acid weight yield per gram of organic carbon in the range of 0.25-0.30.

In some embodiments, the open bioreactor culture may achieve a lipid productivity rate in the range of 0.1-0.2 g/L. In some embodiments, the open bioreactor culture may achieve a lipid productivity rate in the range of 0.2-0.3 g/L. In some embodiments, the open bioreactor culture may achieve a lipid productivity rate in the range of 0.3-0.4 g/L. In some embodiments, the open bioreactor culture may achieve a lipid productivity rate in the range of 0.4-0.5 g/L. In some embodiments, the bag bioreactor culture may achieve a lipid productivity rate in the range of 0.5-0.6 g/L. In some embodiments, the open bioreactor culture may achieve a lipid productivity rate in the range of 0.6-0.7 g/L. In some embodiments, the open bioreactor culture may achieve a lipid productivity rate in the range of 0.7-0.8 g/L. In some embodiments, the open bioreactor culture may achieve a lipid productivity rate in the range of 0.8-0.9 g/L. In some embodiments, the open bioreactor culture may achieve a lipid productivity rate in the range of 0.9-1.0 g/L.

In some embodiments, the open bioreactor culture may achieve an average biomass productivity rate in the range of 1-2 g/L/day. In some embodiments, the open bioreactor culture may achieve an average biomass productivity rate in the range of 2-3 g/L/day. In some embodiments, the open bioreactor culture may achieve an average biomass productivity rate in the range of 3-4 g/L/day. In some embodiments, the open bioreactor culture may achieve an average biomass productivity rate in the range of 4-5 g/L/day. In some embodiments, the bag bioreactor culture may achieve an average biomass productivity rate in the range of 5-6 g/L/day. In some embodiments, the open bioreactor culture may achieve an average biomass productivity rate in the range of 6-7 g/L/day. In some embodiments, the open bioreactor culture may achieve an average biomass productivity rate in the range of 7-8 g/L/day. In some embodiments, the open bioreactor culture may achieve an average biomass productivity rate in the range of 8-9 g/L/day. In some embodiments, the open bioreactor culture may achieve an average biomass productivity rate in the range of 9-10 g/L/day.

In some embodiments, the open bioreactor culture may achieve an average biomass productivity rate in the range of 10-15 g/L/day. In some embodiments, the open bioreactor culture may achieve an average biomass productivity rate in the range of 15-20 g/L/day. In some embodiments, the open bioreactor culture may achieve an average biomass productivity rate in the range of 20-30 g/L/day. In some embodiments, the open bioreactor culture may achieve an average biomass productivity rate in the range of 30-40 g/L/day. In some embodiments, the open bioreactor culture may achieve an average biomass productivity rate in the range of 40-50 g/L/day. In some embodiments, the open bioreactor culture may achieve an average biomass productivity rate in the range of 50-60 g/L/day. In some embodiments, the open bioreactor culture may achieve an average biomass productivity rate in the range of 60-70 g/L/day. In some embodiments, the open bioreactor culture may achieve an average biomass productivity rate in the range of 70-80 g/L/day.

In some embodiments, the microalgae culture may be cultured at a residual acetate range of 0.1-1 g/L. In some embodiments, the microalgae culture may be cultured at a residual acetate range of 1-10 g/L. In some embodiments, the microalgae culture may be cultured at a residual acetate range of 10-20 g/L. In some embodiments, the microalgae culture may be cultured at a residual acetate range of 20-40 g/L. In some embodiments, the microalgae culture may be cultured at a residual acetate range of 40-60 g/L. In some embodiments, the microalgae culture may be cultured at a residual acetate range of 60-80 g/L. In some embodiments, the microalgae culture may be cultured at a residual acetate range of 80-100 g/L.

In some embodiments, the nitrogen source for the culture may comprise monosodium glutamate (MSG), ammonia, ammonium (e.g., ammonium hydroxide, ammonium phosphate, ammonium acetate), urea, nitrates, glycine or a combination thereof. In some embodiments, the aqueous culture medium may comprise trace metals, vitamins, minerals, and combinations thereof. In some embodiments, the culture may comprise an organic carbon source in addition to acetic acid and acetate, and consist of at least one selected from the group comprising: glycerol, glucose, sucrose, fructose, molasses, other suitable sugars, other suitable refined industrial by-product streams, or a combination thereof. In some embodiments, the culture may comprise an initial concentration of acetate and receive a subsequent supply of acetic acid as the organic carbon source. In some embodiments, a portion of the acetic acid used in the method may be replaced by other organic salts or acids such as lactic acid, propionic acid, malic acid, succinic acid, and combinations thereof.

In some embodiments, acetic acid may be used to stabilize a medium that is fed into a culture operating in a continuous culturing mode. In some embodiments, the continuous culturing mode may comprise a single stage (i.e., growth through biomass accumulation and cell division, or lipid accumulation) instead of two or more stages focused on either growth in one stage and accumulation of a metabolite (e.g., lipids) in another stage or stages. In some embodiments, the continuous mode may comprise nitrogen sufficient conditions. In some embodiments, the continuous mode may comprise nitrogen deficient conditions. In some embodiments, the supply of acetic acid may be terminated prior to harvesting the organisms to induce an increase in culture pH.

In some embodiments, cultures in different volumes or different bioreactors may have the same nitrogen source. In some embodiments, cultures in different volumes or different bioreactors may have different nitrogen sources. In some embodiments, cultures in different volumes or different bioreactors may have the same organic carbon source. In some embodiments, cultures in different volumes or different bioreactors may have different organic carbon sources. In some embodiments, cultures in different volumes or different bioreactors may have the same culturing temperature. In some embodiments, cultures in different volumes or different bioreactors may have different culturing temperatures. In some embodiments, cultures in different volumes or different bioreactors may have the same culture pH value. In some embodiments, cultures in different volumes or different bioreactors may have different culture pH values. In some embodiments, cultures in different volumes or different bioreactors may have the same inoculation density. In some embodiments, cultures in different volumes or different bioreactors may have different inoculation densities.

In some embodiments, the Aurantiochytrium may be transferred between different bioreactors of increasing volume multiple times in the process of scaling the volume of the culture up over the life of the culture. In some embodiments, the first culturing stage may occur in a flask. In some embodiments, a culture in a flask may be transferred to a bag bioreactor of a volume larger than the flask for the second culturing stage. In some embodiments, a culture in a bag bioreactor may be transferred to an open bioreactor of volume larger than the bag bioreactor for the third stage of culturing.

In some embodiments, the flask bioreactor may be sterile and maintain the culture of Aurantiochytrium in axenic conditions. In some embodiments, the culture of Aurantiochytrium may be maintained as axenic during transfer to the bag bioreactor. In some embodiments, the bag bioreactor may be sterile and maintain the culture of Aurantiochytrium in axenic conditions. In some embodiments, the culture of Aurantiochytrium may not be axenic, and may be cultured in non-axenic conditions. In some embodiments, the flask may be non-sterile. In some embodiments, the bag bioreactor may be non-sterile. The open bioreactor may be any bioreactor that is not closed including, but not limited to, a trough, a tank, a pond, and a raceway pond.

In some embodiments, the Aurantiochytrium biomass may be harvested from the culture and separated from the aqueous culture medium by any suitable method known in the art such as, but not limited to, filtration, centrifugation, settling, decanting, dissolved air flotation, and skimming. In some embodiments, the culture density may be high enough that the biomass is not separated from the aqueous culture medium before further processing. In some embodiments, the Aurantiochytrium biomass may be further processed to isolate the oil from the biomass by any suitable method known in the art such as, but not limited to, mechanical extraction, solventless extraction, extraction with a solvent, and supercritical carbon dioxide extraction. In some embodiments, the Aurantiochytrium biomass or isolated oil may be further processed to produce a suitable processed product by methods known in the art such as, but not limited to, pasteurization, drying, cell disruption (e.g., milling, pressing), cell lysing, hydrothermal liquefaction, esterification, winterization, refining, urea crystallization, bleaching, deodorizing, distillation, and hydrotreatment.

### Traditional Fermenter Embodiment

In some embodiments, the Aurantiochytrium may be cultured with at least one of acetate and acetic acid in a closed sterile fermenter in an aqueous culture medium. In some embodiments, the closed sterile (axenic) fermenter may be constructed from glass, stainless steel, or any other suitable material. In some embodiments, closed sterile fermenter culture may comprise a culture temperature in the range of 20° C. to 40° C., a culture pH in the range of 4-9, and an initial inoculation density of 0.1-10.0 g/L. In some embodiments, the closed sterile fermenter culture may achieve a culture density of up to 500 g/L. In some embodiments, the closed sterile fermenter culture may achieve a biomass weight yield per gram of organic carbon of up to 0.55. In some embodiments, the closed sterile fermenter culture may achieve an Omega-3 fatty acid weight yield per gram of organic carbon of up to 0.30. In some embodiments, the closed sterile fermenter culture may achieve a lipid productivity rate of up to 1.0 g/L/hr. In some embodiments, the closed sterile fermenter culture may achieve an average biomass productivity rate of up to 60 g/L/day. In some embodiments, the closed sterile fermenter culture may receive an organic carbon supply comprising at least one of acetate and acetic acid as described throughout the specification, such as for the flask culture, bag bioreactor culture, and open bioreactor culture.

In some embodiments, the culture process may comprise a combination of culturing in stages with sterilized bioreactors and non-sterilized bioreactors. In some embodiments, an inoculum culture may be produced in a sterilized bioreactor where cell division and biomass accumulation primarily occurs at a low C:N ratio, and then is transferred to a sterilized or non-sterilized bioreactor of a larger volume as a culture comprise a percent solids in the range of 1-20%. In some embodiments, the inoculated culture may have a percent solids in the range of 1-5%. In some embodiments, the inoculated culture may have a percent solids in the range of 5-10%. In some embodiments, the inoculated culture may have a percent solids in the range of 10-15%. In some embodiments, the inoculated culture may have a percent solids in the range of 15-20%. In some embodiments, the final stage of culturing in a sterilized or non-sterilized bioreactor may comprise nitrogen supplied as a batch. In some embodiments, the final stage of culturing in a sterilized or non-sterilized bioreactor may comprise nitrogen limited conditions and primarily the accumulation of lipids.

In some embodiments, the culture process may comprise a combination of culturing in stages with sterilized bioreactors and non-sterilized bioreactors. In some embodiments, an inoculum culture may be produced in a sterilized bioreactor where cell division and biomass accumulation primarily occurs at a low C:N ratio, and then is transferred to a sterilized or non-sterilized bioreactor of a larger volume as a culture comprise a percent solids in the range of 1-10%. In some embodiments, the inoculated culture may have a percent solids in the range of 1-2%. In some embodiments, the inoculated culture may have a percent solids in the range of 2-3%. In some embodiments, the inoculated culture may have a percent solids in the range of 3-4%. In some embodiments, the inoculated culture may have a percent solids in the range of 4-5%. In some embodiments, the inoculated culture may have a percent solids in the range of 5-7%. In some embodiments, the inoculated culture may have a percent solids in the range of 7-10%. In some embodiments, the final stage of culturing in a sterilized or non-sterilized bioreactor may comprise nitrogen supplied as a batch or supplied as a feed with the organic carbon. In some embodiments, the final stage of culturing in a sterilized or non-sterilized bioreactor may comprise nitrogen sufficient conditions and the concurrent production of cell growth for biomass accumulation and lipid accumulation.

In one non-limiting embodiment, an inoculum culture may be produced in a sterilized bioreactor and used to inoculate a non-sterilized bioreactor operating as a semi-batch non-axenic process to primarily produce lipid accumulation with little to no cell growth and biomass accumulation. In another non-limiting embodiment, an inoculum culture may be produced in a sterilized bioreactor and used to inoculate a non-sterilized bioreactor operating as a semi-batch non-axenic process to produce both biomass accumulation through cell growth and lipid accumulation concurrently. In another non-limiting embodiment, a sterilized or non-sterilized bioreactor may operate as a single phase continuous culture, wherein single phase is defined as producing only biomass accumulation through growth, only lipid accumulation, or both biomass accumulation through growth and lipid accumulation concurrently. In another non-limiting embodiment, an inoculum culture may be produced in a sterilized bioreactor and used to inoculate a sterilized bioreactor operating as a semi-batch axenic process with a first stage comprising primarily biomass accumulation through cell growth in low C:N conditions and a second stage comprising primarily lipid accumulation in nitrogen limited conditions.

### EXAMPLES

Embodiments of the invention are exemplified and additional embodiments are disclosed in further detail in the following Examples, which are not in any way intended to limit the scope of any aspect of the invention described herein. The exemplary strain HS399 was identified through genetic analysis as having high similarities to species of Aurantiochytrium, and was isolated from an aquatic mangrove environment with characteristics similar to where literature has identified species of Schizochytrium have previously been isolated from.

### Example 1

In one experiment Aurantiochytrium sp. (HS399) was cultured in method utilizing acetate and acetic acid as an organic carbon source to produce biomass with the profile shown in Table 1.

TABLE 1

| | |
|---|---|
| Moisture by Forced Draft | 4.79% |
| Oven | |
| Protein (Kjeltec) | 28.98% |
| Ash | 10.09% |
| Carbohydrates (calculated) | 16.05% |
| Crude fat by acid hydrolysis | 44.35% |
| C4:0 (Butyric) | |
| C12:0 (Lauric Acid) | 0.12% |
| C14:0 Myristic | 1.33% |
| C15:0 (Pentadecanoic acid) | 0.12% |
| C16:0 (Palmitic Acid) | 20.64% |
| C17:0 (Margaric Acid) | 0.05% |
| C18:0 (Stearic Acid) | 0.66% |
| C18:3, Total (Linolenic Acid + | 0.07% |
| isomers) | |
| C20:3 Omega 9, Total | 0.04% |
| (Eicosatrienoic Acid) | |
| C20:5 Omega 3 | 0.10% |
| (Eicosapentaenoic Acid) | |
| C22:5 Total | 2.49% |
| (Docosapentaenoic Acid) | |
| C22:6 Omega 3 | 12.03% |
| (Docosahexaenoic) | |
| Total Omega 3 Isomers | 12.42% |
| Total Omega 5 Isomers | <0.01% |
| Total Omega 6 Isomers | 2.56% |
| Total Omega 7 Isomers | 0.05% |
| Total Omega 9 Isomers | 0.07% |
| Total Monounsaturated Fatty | 0.24% |
| Acids | |
| Total Polyunsaturated Fatty | 14.99% |
| Acids | |
| Total Saturated Fatty Acids | 23.07% |
| Total Trans Fatty Acids | 0.05% |
| Total Fat as Triglycerides | 40.09% |
| Total Fatty Acids | 38.35% |
| C16:DHA Ratio | 1.72 |
| DPA:DHA Ratio | 0.21 |
| EPA:DHA Ratio | 0.0083 |

### Example 2

In one experiment Aurantiochytrium sp. (HS399) was cultured in method utilizing acetate and acetic acid as an organic carbon source to produce biomass with the profiles shown in Tables 2-3.

TABLE 2

| | Whole | Whole | Whole | Whole |
|---|---|---|---|---|
| | Biomass | Biomass | Biomass | Biomass |
| | Profile 1 | Profile 2 | Profile 3 | Profile 4 |
| Moisture by | 2.84% | 1.44% | 4.79% | 2.37% |
| Forced Draft Oven | | | | |
| Protein (Kjeltec) | 10.08% | 10.34% | 28.98% | |
| Alanine | 0.51% | 0.54% | 0.99% | <0.10% |
| Arginine | 0.46% | 0.42% | 0.75% | <0.10% |
| Aspartic Acid | 1.12% | 1.12% | | |
| Cystine | 0.13% | 0.12% | 0.04% | <0.10% |
| Glutamic Acid | 0.99% | 1.04% | | |
| Glycine | 0.40% | 0.41% | 0.32% | <0.10% |
| Histidine | 0.18% | 0.20% | 0.27% | <0.10% |
| Isoleucine | 0.37% | 0.35% | 0.34% | <0.10% |
| Leucine | 0.62% | 0.59% | 0.60% | <0.10% |
| Lysine | 0.48% | 0.47% | 0.95% | <0.10% |
| Methionine | 2.37% | 2.28% | 0.34% | <0.10% |
| Phenylalanine | 0.40% | 0.36% | 0.49% | <0.10% |
| Proline | 0.27% | 0.28% | 0.56% | <0.10% |
| Serine | 0.43% | 0.42% | 0.82% | <0.10% |
| Threonine | 0.44% | 0.42% | 0.55% | |
| Tryptophan | 0.15% | 0.13% | 0.14% | <0.10% |
| Tyrosine | 0.28% | 0.27% | 0.41% | <0.10% |
| Valine | 0.48% | 0.47% | 0.45% | <0.10% |
| Ash | 8.08% | 7.17% | 10.09% | 6.79% |
| Carbohydrates | 11.91% | 11.51% | 16.05% | 9.36 g/100 g |
| (calculated) | | | | |
| Total Vitamin A | 1,250 IU/100 g | 1,130 IU/100 g | <25 IU/100 g | 89.50 IU/100 g |
| Beta-Carotene | 1,250 IU/100 g | 1,130 IU/100 g | 0.00 IU/100 g | |
| | (0.00074985 g/ | (0.000677864 g/ | | |
| | 100 g) | 100 g) | | |
| Retinal | <294 IU/100 g | <293 IU/100 g | — | |
| Biotin | 0.0944 mg/100 g | 0.198 mg/100 g | 166.88 mcg/ | |
| | | | 100 g | |
| Vitamin B1- | 0.0715 mg/100 g | 0.463 mg/100 g | — | 5.5 mg/100 g |
| Thiamine | | | | |
| Hydrochloride | | | | |
| Vitamin B2- | 0.670 mg/100 g | 1.49 mg/100 g | 5.71 mg/100 g | |
| Riboflavin | | | | |
| Vitamin B3-Total | 1.12 mg/100 g | 7.87 mg/100 g | 2.92 mg/100 g | 5.32 mg/100 g |
| Niacin | | | | |
| Vitamin B5- | NA | 0.341 mg/100 g | 0.89 mg/100 g | 0.64 mg/100 g |
| Pantothenic Acid | | | | |
| Vitamin B6- | 0.202 mg/100 g | 0.354 mg/100 g | 1.40 mg/100 g | 44.11 mcg/100 g |
| Pyridoxine | | | | |
| Hydrochloride | | | | |
| Vitamin B9-Total | NA | 1.53 mg/100 g | — | 8.44 mcg/100 g |
| Folic Acid | | | | |
| Vitamin B12- | 3.03 μg/100 g | 0.966 μg/100 g | 10.66 mcg/100 g | 4.78 mcg/100 g |
| Cyanocobalamin | | | | |
| Vitamin C (as | <0.440 mg/ | <0.440 mg/100 g | 54.12 mg/100 g | <1.0 mg/100 g |
| Ascorbic Acid) by | 100 g | | | |
| HPLC | | | | |
| Cadmium | <0.010 mg | 0.010 mg/kg | <0.020 ppm | <0.020 ppm |
| Lead | 0.300 mg/kg | 0.106 mg/kg | <0.020 ppm | <0.020 ppm |
| Mercury | <0.010 mg/kg | <0.010 mg/kg | <0.010 ppm | <0.025 ppm |
| Calcium | 0.168% | 0.154% | | |
| Phosphorus | 0.25% | 0.86% | | |
| Potassium | 0.14% | 0.54% | | |
| Selenium | <0.1 ppm | <0.1 ppm | | |
| Zinc | 6.5 ppm | 16 ppm | | |
| Arsenic | 0.158 mg/mg | 0.141 mg/kg | 0.287 ppm | |
| Crude fat by acid | 71.55% | 74.21% | | |
| hydrolysis | | | | |
| C12:0 (Lauric | 0.11% | 0.12% | 0.12% | 0.10% |
| Acid) | | | | |
| C14:0 Myristic | 2.24% | 2.19% | 1.33% | 2.99% |
| C15:0 | 0.13% | 0.12% | 0.12% | 0.12% |
| (Pentadecanoic | | | | |
| acid) | | | | |
| C16:0 (Palmitic | 34.40% | 34.13% | 20.64% | 45.32% |
| Acid) | | | | |
| C17:0 (Margaric | 0.05% | 0.05% | 0.05% | 0.04% |
| Acid) | | | | |
| C18:0 (Stearic | 1.00% | 0.98% | 0.66% | 1.03% |
| Acid) | | | | |
| C18:3, Total | 0.08% | 0.08% | 0.07% | 0.03% |
| (Linolenic Acid + | | | | |
| isomers) | | | | |
| C20:3 Omega 9, | 0.07% | 0.08% | 0.04% | 0.07% |
| Total | | | | |
| (Eicosatrienoic | | | | |
| Acid) | | | | |
| C20:5 Omega 3 | 0.13% | 0.12% | 0.10% | 0.05% |
| (Eicosapentaenoic | | | | |
| Acid) | | | | |
| C22:5 Total | 4.30% | 4.77% | 2.49% | — |
| (Docosapentaenoic | | | | |
| Acid) | | | | |
| C22.6 Omega 3 | 20.80% | 23.01% | 12.03% | 22.96% |
| (Docosahexaenoic) | | | | |
| Total Omega 3 | 21.28% | 23.51% | 12.42% | — |
| Isomers | | | | |
| Total Omega 5 | <0.01% | <0.01% | | — |
| Isomers | | | | |
| Total Omega 6 | 4.41% | 4.90% | 2.56% | — |
| Isomers | | | | |
| Total Omega 7 | 0.06% | 0.05% | 0.05% | — |
| Isomers | | | | |
| Total Omega 9 | 0.03% | 0.06% | 0.07% | — |
| Isomers | | | | |
| Total | 0.25% | 0.27% | 0.24% | 0.46% |
| Monounsaturated | | | | |
| Fatty Acids | | | | |
| Total | 25.70% | 28.41% | 14.99% | 23.53% |
| Polyunsaturated | | | | |
| Fatty Acids | | | | |
| Total Saturated | 38.14% | 37.78% | 23.07% | 53.08% |
| Fatty Acids | | | | |
| Total Trans Fatty | 0.09% | 0.09% | 0.05% | 0.03% |
| Acids | | | | |
| Total Fat as | 67.09% | 69.54% | 40.09% | — |
| Triglycerides | | | | |
| Total Fatty Acids | 64.18% | 66.55% | 38.35% | 80.98% |
| C16:DHA Ratio | 1.65 | 1.48 | 1.72 | 1.97 |
| DPA:DHA Ratio | 0.21 | 0.21 | 0.21 | — |
| EPA:DHA Ratio | 0.0063 | 0.0052 | 0.0083 | 0.002 |

TABLE 3

| | Whole Biomass | Whole Biomass |
|---|---|---|
| | Profile 5 | Profile 6 |
| Moisture by | 2.84% | 5.21% |
| Forced Draft Oven | | |
| Protein (Kjeltec) | 10.08% | 33.55% |
| Alanine | 0.51% | |
| Arginine | 0.46% | |
| Aspartic Acid | 1.12% | |
| Cystine | 0.13% | |
| Glutamic Acid | 0.99% | |
| Glycine | 0.40% | |
| Histidine | 0.18% | |
| Isoleucine | 0.37% | |
| Leucine | 0.62% | |
| Lysine | | |
| Methionine | 2.37% | |
| Phenylalanine | 0.40% | |
| Proline | 0.27% | |
| Serine | 0.43% | |
| Threonine | 0.44% | |
| Tryptophan | 0.15% | |
| Tyrosine | 0.28% | |
| Valine | 0.48% | |
| Ash | 8.08% | 14.32% |
| Carbohydrates | 11.91% | |
| (calculated) | | |
| Total Vitamin A | 1250 IU/100 g | |
| Beta-Carotene | 1250 IU/100 g | |
| Retinal | <294 IU/100 g | |
| Biotin | 0.0944 mg/100 g | |
| Vitamin B1- | 0.0715 mg/100 g | |
| Thiamine | | |
| Hydrochloride | | |
| Vitamin B2- | 0.670 mg/100 g | |
| Riboflavin | | |
| Vitamin B3-Total | 1.12 mg/100 g | |
| Niacin | | |
| Vitamin B5- | | |
| Pantothenic Acid | | |
| Vitamin B6- | 0.202 mg/100 g | |
| Pyridoxine | | |
| Hydrochloride | | |
| Vitamin B12- | 3.03 ug/100 g | |
| Cyanocobalamin | | |
| Vitamin C (as | <0.440 mg/100 g | |
| Ascorbic Acid) by | | |
| HPLC | | |
| Cadmium | <0.010 mg/kg | |
| Lead | 0.300 mg/kg | |
| Mercury | <0.010 mg/kg | |
| Calcium | 0.168% | |
| Phosphorus | 0.25% | |
| Potassium | 0.14% | |
| Selenium | <0.1 ppm | |
| Zinc | 6.5 ppm | |
| Arsenic | 0.158 mg/kg | |
| Crude fat by acid | 71.55% | 34.91% |
| hydrolysis | | |
| C12:0 (Lauric | 0.11% | 0.08% |
| Acid) | | |
| C14:0 Myristic | 2.24% | 0.77% |
| C15:0 | 0.13% | 0.06% |
| (Pentadecanoic | | |
| acid) | | |
| C16:0 (Palmitic | 34.40% | 14.59% |
| Acid) | | |
| C17:0 (Margaric | 0.05% | 0.03% |
| Acid) | | |
| C18:0 (Stearic | 1.00% | 0.50% |
| Acid) | | |
| C18:3, Total | 0.08% | 0.05% |
| (Linolenic Acid + | | |
| isomers) | | |
| C20:3 Omega 9, | 0.07% | 0.03% |
| Total | | |
| (Eicosatrienoic | | |
| Acid) | | |
| C20:4 (ARA) | 0.03% | 0.03% |
| C20:5 Omega 3 | 0.13% | 0.13% |
| (Eicosapentaenoic | | |
| Acid) | | |
| C22:5 Total | 4.30% | 2.07% |
| (Docosapentaenoic | | |
| Acid) | | |
| C22:6 Omega 3 | 20.80% | 11.48% |
| (Docosahexaenoic) | | |
| Total Omega 3 | 21.28% | 11.86% |
| Isomers | | |
| Total Omega 6 | 4.41% | 2.11% |
| Isomers | | |
| Total Omega 7 | 0.06% | 0.04% |
| Isomers | | |
| Total Omega 9 | 0.03% | 0.11% |
| Isomers | | |
| Total | 0.25% | 0.22% |
| Monounsaturated | | |
| Fatty Acids | | |
| Total | 25.70% | 13.97% |
| Polyunsaturated | | |
| Fatty Acids | | |
| Total Saturated | 38.14% | 16.12% |
| Fatty Acids | | |
| Total Trans Fatty | 0.09% | 0.05% |
| Acids | | |
| Total Fat as | 67.09% | 31.72% |
| Triglycerides | | |
| Total Fatty Acids | 64.18% | 30.37% |
| C16:DHA Ratio | 1.654 | 1.271 |
| DPA:DHA Ratio | 0.207 | 0.180 |
| EPA:DHA Ratio | 0.006 | 0.011 |
| ARA:DHA ratio | 0.001 | 0.003 |

### Example 3

In one experiment Aurantiochytrium sp. (HS399) was cultured in method utilizing acetate and acetic acid as an organic carbon source to produce biomass, whereupon a solventless extraction process was performed to produce the crude oil with the exemplary profiles in Table 4, and the residual biomass with the exemplary profiles in Table 5. The residual biomass profile 3 resulted from a French press process using a fiber to aid separation of oil and biomass.

TABLE 4

| | Crude Oil | Crude Oil | Crude Oil |
|---|---|---|---|
| | Profile 1 | Profile 2 | Profile 3 |
| Moisture by | 2.90% | — | — |
| Forced Draft Oven | | | |
| Ash | <0.1% | — | — |
| C12:0 (Lauric | 0.16% | 0.10% | 0.09% |
| Acid) | | | |
| C14:0 Myristic | 3.20% | 2.93% | 2.86% |
| C15:0 | 0.13% | 0.18% | 0.18% |
| (Pentadecanoic | | | |
| acid) | | | |
| C16:0 (Palmitic | 48.01% | 49.15% | 47.49% |
| Acid) | | | |
| C17:0 (Margaric | 0.05% | 0.06% | 0.07% |
| Acid) | | | |
| C18:0 (Stearic | 1.39% | 1.35% | 0.31% |
| Acid) | | | |
| C18:3, Total | 0.04% | 0.10% | 0.10% |
| (Linolenic Acid + | | | |
| isomers) | | | |
| C20:3 Omega 9, | 0.11% | 0.09% | 0.10% |
| Total | | | |
| (Eicosatrienoic | | | |
| Acid) | | | |
| C20:4 (ARA) | 0.04% | 0.04% | 0.03% |
| C20:5 Omega 3 | 0.14% | 0.13% | 0.13% |
| (Eicosapentaenoic | | | |
| Acid) | | | |
| C22:5 Total | 6.66% | 6.15% | 6.04% |
| (Docosapentaenoic | | | |
| Acid) | | | |
| C22:6 Omega 3 | 32.03% | 28.74% | 27.91% |
| (Docosahexaenoic) | | | |
| Total Omega 3 | 32.67% | 29.52% | 28.69% |
| Isomers | | | |
| Total Omega 6 | 6.82% | 6.12% | 6.02% |
| Isomers | | | |
| Total Omega 7 | 0.05% | 0.05% | 0.05% |
| Isomers | | | |
| Total Omega 9 | 0.06% | 0.03% | 0.01% |
| Isomers | | | |
| Total | 0.44% | 0.27% | 0.30% |
| Monounsaturated | | | |
| Fatty Acids | | | |
| Total | 39.66% | 35.64% | 34.71% |
| Polyunsaturated | | | |
| Fatty Acids | | | |
| Total Saturated | 53.09% | 54.07% | 52.60% |
| Fatty Acids | | | |
| Total Trans Fatty | 0.08% | 0.08% | 0.10% |
| Acids | | | |
| Total Fat as | 97.47% | 94.14% | 91.67% |
| Triglycerides | | | |
| Total Fatty Acids | 93.28% | 90.07% | 87.70% |
| C16:DHA Ratio | 1.50 | 1.710 | 1.712 |
| DPA:DHA Ratio | 0.208 | 0.214 | 0.216 |
| EPA:DHA Ratio | 0.004 | 0.004 | 0.005 |
| ARA:DHA ratio | 0.001 | 0.001 | 0.001 |
| Monoglycerides | 1.25% | — | — |
| Diglycerides | 2.48% | — | — |
| Triglycerides | 94.63% | — | — |
| Glycerol | <1% | — | — |

TABLE 5

| | Residual | Residual | Residual |
|---|---|---|---|
| | Biomass | Biomass | Biomass |
| | Profile 1 | Profile 2 | Profile 3 |
| Moisture by | 12.20% | 7.45% | 4.23% |
| Forced Draft Oven | | | |
| Protein | 41.90% | 48.31% | 18.05% |
| Ash | 17.35% | 19.63% | 13.13% |
| C12:0 (Lauric | 0.06% | 0.05% | 0.06% |
| Acid) | | | |
| C14:0 Myristic | 0.20% | 0.16% | 0.23% |
| C15:0 | 0.02% | 0.01% | 0.01% |
| (Pentadecanoic | | | |
| acid) | | | |
| C16:0 (Palmitic | 3.98% | 3.23% | 3.14% |
| Acid) | | | |
| C17:0 (Margaric | <0.01% | <0.01% | 0.01% |
| Acid) | | | |
| C18:0 (Stearic | 0.14% | 0.10% | 0.17% |
| Acid) | | | |
| C18:3, Total | 0.02% | 0.01% | 0.06% |
| (Linolenic Acid + | | | |
| isomers) | | | |
| C20:3 Omega 9, | <0.01% | 0.01% | 0.01% |
| Total | | | |
| (Eicosatrienoic | | | |
| Acid) | | | |
| C20:5 Omega 3 | 0.05% | 0.04% | 0.01% |
| (Eicosapentaenoic | | | |
| Acid) | | | |
| C22:5 Total | 0.69% | 0.51% | 0.44% |
| (Docosapentaenoic | | | |
| Acid) | | | |
| C22:6 Omega 3 | 3.84% | 2.80% | 1.90% |
| (Docosahexaenoic) | | | |
| Total Omega 3 | 3.96% | 2.89% | 1.99% |
| Isomers | | | |
| Total Omega 6 | 0.71% | 0.53% | 0.6% |
| Isomers | | | |
| Total Omega 7 | <0.01% | 0.01% | 0.03% |
| Isomers | | | |
| Total Omega 9 | 0.04% | 0.03% | 0.14% |
| Isomers | | | |
| Total | 0.06% | 0.06% | 0.2% |
| Monounsaturated | | | |
| Fatty Acids | | | |
| Total | 4.67% | 3.42% | 2.6% |
| Polyunsaturated | | | |
| Fatty Acids | | | |
| Total Saturated | 4.43% | .358% | 3.69% |
| Fatty Acids | | | |
| Total Trans Fatty | 0.02% | 0.01% | 0.03% |
| Acids | | | |
| Total Fat as | 9.58% | 7.38% | 6.81% |
| Triglycerides | | | |
| Total Fatty Acids | 9.18% | 7.06% | 6.52% |
| C16:DHA Ratio | 1.036 | 1.154 | 1.653 |
| DPA:DHA Ratio | 0.180 | 0.182 | 0.232 |
| EPA:DHA Ratio | 0.013 | 0.014 | 0.005 |

The crude oil was then subjected to a winterization process using a polar solvent and lower temperatures to precipitate compounds from the crude oil. The multiple exemplary profiles of the DHA rich fraction, as well as an exemplary profile of the palmitic acid rich fraction resulting from the winterization process in weight percentage are shown in Table 6.

TABLE 6

| | | | | Palmitic |
|---|---|---|---|---|
| | DHA Rich | DHA Rich | DHA Rich | Acid Rich |
| Analytes | Fraction (1) | Fraction (2) | Fraction (3) | Fraction |
| C12:0 | 0.13% | 0.13% | 0.15% | 0.08% |
| C14:0 | 3.71% | 3.62% | 4.04% | 2.55% |
| C15:0 | 0.16% | 0.17% | 0.19% | 0.21% |
| C16:0 | 31.08% | 31.85% | 30.37% | 59.23% |
| C16:1 | 0.15% | 0.13% | 0.15% | 0.08% |
| C17:0 | 0.05% | 0.06% | 0.06% | 0.11% |
| C18:0 | 1.43% | 1.54% | 1.32% | 1.08% |
| C18:1 | 0.66% | 0.74% | 0.62% | 0.04% |
| C18:2 | 0.34% | 0.42% | 0.19% | 0.07% |
| C18:3 | 0.18% | 0.18% | 0.19% | 0.12% |
| C20:0 | 0.23% | 0.24% | 0.25% | 0.14% |
| C20:4n6 | 0.07% | 0.07% | 0.07% | 0.03% |
| C20:5n3 | 0.23% | 0.22% | 0.25% | 0.03% |
| C22:0 | 0.10% | 0.10% | 0.10% | 0.27% |
| C22:5n6 | 8.76% | 8.69% | 8.60% | 6.53% |
| C22:6n3 | 38.51% | 38.75% | 38.81% | 24.85% |
| Total Omega-3 | 39.92% | 39.95% | 40.09% | 25.39% |
| Isomers | | | | |
| Total Omega-6 | 9.34% | 9.31% | 9.01% | 6.90% |
| Isomers | | | | |
| Total Omega-7 | 0.11% | 0.08% | 0.12% | — |
| Isomers | | | | |
| Total Omega-9 | 0.26% | 0.36% | 0.20% | 0.08% |
| Isomers | | | | |
| Total | 0.81% | 0.87% | 0.80% | 0.27% |
| Monosaturated | | | | |
| Fatty Acids | | | | |
| Total | 49.31% | 49.31% | 49.16% | 32.29% |
| Polyunsaturated | | | | |
| Fatty Acids | | | | |
| Total Saturated | 36.93% | 37.72% | 35.51% | 63.56% |
| Fatty Acids | | | | |
| Total Fat as | 90.99% | 91.90% | 90.40% | |
| Triglycerides | | | | |
| ARA:DHA ratio | 0.002 | 0.002 | 0.002 | 0.001 |
| DPA:DHA ratio | 0.233 | 0.226 | 0.223 | 0.263 |
| DHA:DPA ratio | 4.284 | 4.434 | 4.487 | 3.81 |
| C16:DHA ratio | 0.807 | 0.797 | 0.758 | 2.38 |
| Cholesterol | 312 mg/ | 346 mg/ | 372 mg/ | |
| | 100 g | 100 g | 100 g | |

### Example 4

An experiment was conducted to determine if Aurantiochytrium sp. HS399 is able to grow using acetate as an organic carbon source. Aurantiochytrium was grown in an acetic acid/pH-auxostat mode in 700 ml bubble column at 27° C. in sterile conditions. The initial culture medium was supplemented and 0.5 g/L sodium acetate and no other organic carbon source. The base medium contained: sodium acetate (1 g/L), monosodium glutamate monohydrate (2.5 g/L) NaCl (12.5 g/L), MgSO₄.7H₂O (2.5 g/L), KCl (0.5 g/L). CaCl₂ (0.1 g/L), KH₂PO₄ (0.125 g/L) vitamins (0.25 ml/L) and trace metal (1.25 ml/L). Trace and vitamins stocks were prepared according to Ashford, et al. 2000. Lipids 35, 1377-1386 The residual acetic acid was maintained at 1±0.5 g/L and the impact of acetate toxicity on the microalgae growth was studied at a pH set point of 5, 4.5 or 7. Cell dry weights and residual acetate concentration in the culture medium were measured daily, and the results are shown in Tables 7 and 8. The results show that Aurantiochytrium was able to grow using acetate as an organic carbon source in cultures with a pH of 5 and above.

TABLE 7
cell dry weights in g/L

| | pH | | | |
|---|---|---|---|---|
| Time (d) | 4.5 | 5.0 | 5.5 | 7.0 |
| 0 | 0.4 | 0.4 | 0.4 | 0.4 |
| 1 | 0.5 ± 0.0 | 2.3 ± 0.1 | 2.7 ± 0.0 | 1.5 ± 2.1 |
| 2 | 0.3 ± 0.1 | 5.0 ± 0.2 | 5.6 ± 0.1 | 2.9 ± 4.1 |
| 3 | 0.2 ± 0.1 | 7.1 ± 0.2 | 8.0 ± 0.0 | 4.1 ± 5.8 |
| 4 | 0.3 ± 0.1 | 8.9 ± 0.1 | 10.2 ± 0.1 | 5.4 ± 7.7 |
| 5 | 0.0 ± 0.0 | 10.0 ± 0.1 | 11.6 ± 0.1 | 6.3 ± 8.9 |

TABLE 8
residual acetate (g/L)

pH

| Time (d) | 4.5 | 5.00 | 5.50 | 7.00 |
|---|---|---|---|---|
| 0 | 0.3 | 0.3 | 0.3 | 0.3 |
| 1 | 2.0 ± 0.1 | 1.6 ± 0.0 | 1.3 ± 0.0 | 0.5 ± 0.7 |
| 2 | 1.9 ± 0.1 | 1.7 ± 0.1 | 1.3 ± 0.1 | 0.4 ± 0.6 |
| 3 | 2.0 ± 0.3 | 1.5 ± 0.1 | 1.2 ± 0.0 | 0.5 ± 0.6 |
| 4 | 1.9 ± 0.3 | 1.5 ± 0.1 | 1.2 ± 0.0 | 0.5 ± 0.7 |
| 5 | 2.0 ± 0.1 | 1.5 ± 0.0 | 1.2 ± 0.0 | 0.5 ± 0.7 |

### Example 5

An experiment was performed to determine the effectiveness of culturing Aurantiochytrium sp. HS399 in a method comprising the use of acetic acid as a carbon source in non-axenic culture conditions. In the first culturing stage four 500 mL axenic flask cultures were inoculated at 0.2 g/L and cultured until the culture density reached 10-20 g/L. The flask cultures received glycerol as an organic carbon source and were cultured at a pH of about 7. In the second culturing stage, the flask cultures were transferred to a single sterile bag bioreactor of a 200 L volume in a sterile manner to maintain the axenic status of the culture. The bag bioreactor culture was cultured from an inoculation density of 0.2 g/L until the culture density reached 10-20 g/L. The bag bioreactor culture received mixture of half glycerol and half acetate as an organic carbon source and was cultured at a pH of about 6.

The bag bioreactor culture was transferred to a non-sterile open raceway pond bioreactor with a volume of about 18,000 L and disposed outdoors. The non-axenic culture was cultured from an inoculation density of about 0.2 g/L until the culture density reached 10-20 g/L. The open bioreactor culture received acetic acid through a pH auxostat system where the pH set point was 5.5. Results of the culturing in the open bioreactor stage are shown in Tables 9, 10, 11, and 12.

TABLE 9

| Time (d) | Cell Dry Weight (g/L) | Lipids (g/L) |
|---|---|---|
| 0.0 | 0.3 | 0.2 |
| 0.2 | 0.4 | |
| 0.7 | 2.8 | 2.4 |
| 1.2 | 4.8 | |
| 1.7 | 9.4 | |
| 2.0 | 10.3 | 8.0 |
| 2.2 | 11.1 | |

TABLE 10

| | Cumulative Acetic | Residual Acetic |
|---|---|---|
| Time (d) | Acid Input (g/L) | Acid (g/L) |
| 0.0 | 0.5 | 0.7 |
| 0.9 | 13.4 | 1.6 |
| 1.3 | 19.2 | |
| 1.9 | 34.6 | |
| 1.9 | 35.9 | 1.7 |
| 2.1 | 38.8 | |

TABLE 11

| Time (d) | Lean Biomass (g/L) | Total Fatty Acids (g/L) | DHA (g/L) |
|---|---|---|---|
| 0.0 | 0.1 | 0.1 | 0.0 |
| 0.9 | 2.0 | 1.4 | 0.4 |
| 1.9 | 2.6 | 5.6 | 1.7 |
| 2.0 | 2.3 | 6.2 | 1.9 |

TABLE 12

| Time (d) | Aerobic bacteria count (CFU/mL) |
|---|---|
| 0.0 | 1.1E+04 |
| 0.9 | 2.1E+04 |
| 1.9 | 2.2E+04 |

As show in Tables 9-12, the culturing method using acetate as an organic carbon source resulted in successful microalgae growth, lipid accumulation, and management of the bacteria population in open outdoor conditions. This exemplifies that methods of the invention can be performed in non-axenic systems while maintaining the increase in bacterial populations to less than 500%, more typically less than 300%, such as less than 250%, or less than 225%, or even restricting such growth to about 200% or less of the starting bacterial concentration and/or population in the culture.

A summary of multiple replicates of this method are shown in Table 13.

TABLE 13

| Experimental Run | 1 | 2 | 3 | 4 | Mean values |
|---|---|---|---|---|---|
| Duration (d) | 2.17 | 1.99 | 1.89 | 2.00 | 2.01 ± 0.12 |
| Final DW (g/L) | 11.10 | 10.5 | 8.68 | 9.28 | 9.89 ± 1.12 |
| Avg. productivity | 5.12 | 5.26 | 4.59 | 4.64 | 4.88 ± 0.37 |
| (g/L-d) | | | | | |
| Acetic acid yield | 0.30 | 0.29 | 0.27 | 0.27 | 0.28 ± 0.02 |
| (g of biomass | | | | | |
| produced/g of | | | | | |
| acetic acid | | | | | |
| consumed) | | | | | |
| Final Aerobic | 2.20 × | 4.40 × | 1.75 × | 3.30 × | 9.54 × 10⁶ ± |
| bacterial count | 10⁴ | 10⁶ | 10⁶ | 10⁷ | 1.58 × 10⁷ |
| (CFU/mL) | | | | | |
| Final lipid | 77.05 | 73.05 | 75 | 74.7 | 75 ± 1.64 |
| content (% DW) | | | | | |
| Final protein | 8.5 | 9.5 | 10 | 10.1 | 9.53 ± 0.73 |
| content (% DW) | | | | | |
| Final total fatty | 59.97 | 44.14 | 69.50 | 68.57 | 60.55 ± 11.75 |
| acid (% DW) | | | | | |
| Final DHA | 29.88 | 30.77 | 30.92 | 30.65 | 30.56 ± 0.46 |
| (% TFA) | | | | | |
| Final DHA:C:16 | 0.53 | 0.59 | 0.57 | 0.56 | 0.56 ± 0.03 |
| ratio | | | | | |

### Example 6

An experiment was performed to determine if Aurantiochytrium sp. HS399 can grow using acetate as an organic carbon source and ammonia as a nitrogen source. Aurantiochytrium was grown in an acetic acid/pH-auxostat mode in 700 ml bubble column at 27° C. in sterile conditions. The base medium contained: NaCl (12.5 g/L), MgSO₄.7H₂O (2.5 g/L), KCl (0.5 g/L). CaCl₂) (0.1 g/L), KH₂PO₄ (0.125 g/L) vitamins (0.25 ml/L) and trace metal (1.25 ml/L). Trace and vitamins stocks were prepared according to Ashford, et al. 2000. Lipids 35, 1377-1386. A blend of ammonium (hydroxide and acetic acid 1:5 w/w) with slightly higher molar ratio of acetic acid (1:1.1) was prepared with the aim maintain residual acetate present until the end of the pH auxostat. The pH auxostat was set to pH 6.6. The ammonium acetate blend was added to the treatments in order to match the following concentrations: 1×: 1.07 g/L NH₄, 5.68 g/L acetic acid, 3×: 3.21 g/L NH₄, 17.04 g/L acetic acid, 5×: 5.35 g/L NH₄, 28.40 g/L acetic acid, 7×: 7.49 g/L NH₄, 39.76 g/L acetic acid. Cell dry weights and residual culture medium acetate were measured daily, and results are shown in Tables 14, 15, and 16. The results show that Aurantiochytrium was able to grow on the 1× and 3× ammonium acetate blends.

TABLE 14

| | Cell Dry Weight (g/L) | | | |
|---|---|---|---|---|
| Time (d) | 1X NH₄AoC | 3X NH₄AoC | 5X NH₄AoC | 7X NH₄AoC |
| 0 | 0.6 ± 0.0 | 0.6 ± 0.0 | 0.6 ± 0.0 | 0.6 ± 0.0 |
| 1 | 2.2 ± 0.0 | 1.7 ± 0.1 | 0.8 ± 0.2 | 1.0 ± 0.2 |
| 2 | 4.8 ± 0.0 | 3.6 ± 0.3 | 0.7 ± 0.1 | 0.6 ± 0.1 |
| 3 | 7.0 ± 0.1 | 5.2 ± 0.1 | 0.7 ± 0.0 | 0.7 ± 0.0 |
| 4 | 7.9 ± 0.1 | 6.5 ± 0.3 | 0.8 ± 0.1 | 0.8 ± 0.1 |
| 5 | 8.7 ± 0.2 | 6.8 ± 0.3 | 0.3 ± 0.0 | 0.3 ± 0.0 |
| 6 | 9.4 ± 0.1 | 7.5 ± 0.1 | 0.4 ± 0.0 | 0.6 ± 0.1 |

TABLE 15

| | Residual Acetate (g/L) | | | |
|---|---|---|---|---|
| Time (d) | 1X NH₄AoC | 3X NH₄AoC | 5X NH₄AoC | 7X NH₄AoC |
| 0 | 5.8 ± 0.0 | 17.0 ± 0.0 | 28.5 ± 0.5 | 38.5 ± 0.5 |
| 1 | 5.2 ± 0.0 | 17.0 ± 0.0 | 30.0 ± 0.0 | 40.0 ± 0.0 |
| 2 | 3.9 ± 0.0 | 16.5 ± 0.5 | 31.5 ± 0.5 | 42.5 ± 0.5 |
| 3 | 3.0 ± 0.1 | 15.5 ± 0.5 | 33.0 ± 0.0 | 44.5 ± 0.5 |
| 4 | 2.1 ± 0.1 | 14.0 ± 0.0 | 28.0 ± 0.0 | 39.0 ± 0.0 |
| 5 | 1.9 ± 0.0 | 13.5 ± 0.5 | 28.0 ± 1.0 | 38.0 ± 2.0 |
| 6 | 1.9 ± 0.0 | 13.5 ± 0.5 | 29.0 ± 0.0 | 39.5 ± 0.5 |

TABLE 16

| | CytosolicResidual Acetate (g/L) | | | |
|---|---|---|---|---|
| Time (d) | 1X NH₄AoC | 3X NH₄AoC | 5X NH₄AoC | 7X NH₄AoC |
| 0 | 28.6 ± 0.0 | 83.8 ± 0.0 | 140.4 ± 2.5 | 189.6 ± 2.6 |
| 1 | 25.6 ± 0.0 | 83.8 ± 0.0 | 147.8 ± 0.0 | 197.1 ± 0.0 |
| 2 | 19.2 ± 0.0 | 81.3 ± 2.5 | 155.2 ± 2.5 | 209.4 ± 2.5 |
| 3 | 14.8 ± 0.5 | 76.4 ± 2.5 | 162.6 ± 0.0 | 219.2 ± 2.5 |
| 4 | 10.3 ± 0.5 | 69.0 ± 0.0 | 137.9 ± 0.0 | 192.1 ± 0.0 |
| 5 | 9.4 ± 0.0 | 66.5 ± 2.5 | 137.9 ± 4.9 | 187.2 ± 9.9 |
| 6 | 9.4 ± 0.0 | 66.5 ± 2.5 | 142.9 ± 0.0 | 194.6 ± 2.5 |

### Example 7

A experiment was performed to determine if Aurantiochytrium sp. HS399 can grow using acetate as an organic carbon source and different sources of nitrogen. Aurantiochytrium was grown in an acetic acid/pH-auxostat mode in 700 ml bubble column at 27° C. in sterile conditions. The base medium contained: NaCl (12.5 g/L), MgSO₄.7H₂O (2.5 g/L), KCl (0.5 g/L). CaCl₂ (0.1 g/L), KH₂PO₄ (0.125 g/L) vitamins (0.25 ml/L) and trace metal (1.25 ml/L). Trace and vitamins stocks were prepared according to Ashford, et al. 2000. Lipids 35, 1377-1386. A blend of ammonium (hydroxide and acetic acid 1:5 w/w) with slightly higher molar ratio of acetic acid (1:1.1) was prepared with the aim maintain residual acetate present until the end of the pH auxostat. The pH auxostat was set to pH 5.5 or 6.0. The ammonium acetate blend was compared against glutamate at two different equimolar concentrations. Cell dry weights and residual culture medium acetate, and results are shown in Tables 17 and 18. The results showed that Aurantiochytrium could grow with either glutamate or ammonia under an acetic acid pH auxostat system. The results show that Aurantiochytrium was able to grow on both glutamate and ammonia as a nitrogen source under an acetic acid pH auxostat system.

TABLE 17

| | Cell Dry Weight (g/L) | | | |
|---|---|---|---|---|
| | pH 5.5 | pH 5.5 | pH 6.0 | pH 6.0 |
| | 1.1 (g/L) | 2.5 (g/L) | 4.5 (g/L) | 10 (g/L) |
| Time (d) | NH4AoC | MSG | NH4AoC | MSG |
| 0 | 0.2 ± 0.0 | 0.2 ± 0.0 | 0.2 ± 0.0 | 0.2 ± 0.0 |
| 1 | 2.5 ± 0.1 | 2.6 ± 0.0 | 2.6 ± 0.1 | 3.6 ± 0.3 |
| 2 | 4.9 ± 0.1 | 5.2 ± 0.0 | 5.4 ± 0.4 | 6.1 ± 0.1 |
| 3 | 7.5 ± 0.3 | 7.5 ± 0.2 | 7.8 ± 0.1 | 8.1 ± 0.3 |
| 4 | 8.9 ± 0.2 | 8.8 ± 0.4 | 9.8 ± 0.5 | 7.9 ± 2.0 |
| 5 | 10.7 ± 0.3 | 10.4 ± 0.4 | 11.9 ± 0.2 | 9.7 ± 2.7 |
| 6 | 11.8 ± 0.1 | 11.4 ± 0.3 | 13.8 ± 0.2 | 11.9 ± 2.6 |

TABLE 18

Residual Acetate (g/L)

| | pH 5.5 | pH 5.5 | pH 6.0 | pH 6.0 |
|---|---|---|---|---|
| | 1.1 (g/L) | 2.5 (g/L) | 4.5 (g/L) | 10 (g/L) |
| Time (d) | NH4AoC | MSG | NH4AoC | MSG |
| 0 | 0.9 ± 0.0 | 0.4 ± 0.0 | 3.1 ± 0.1 | 0.4 ± 0.0 |
| 1 | 0.1 ± 0.0 | 1.2 ± 0.0 | 2.0 ± 0.1 | 2.2 ± 0.1 |
| 2 | 0.1 ± 0.1 | 1.1 ± 0.0 | 0.9 ± 0.1 | 3.1 ± 0.2 |
| 3 | 0.2 ± 0.0 | 1.1 ± 0.1 | 0.5 ± 0.1 | 2.9 ± 0.0 |
| 4 | 0.3 ± 0.0 | 1.1 ± 0.0 | 0.3 ± 0.0 | 3.3 ± 0.6 |
| 5 | 0.3 ± 0.0 | 1.2 ± 0.1 | 0.3 ± 0.0 | 3.2 ± 0.5 |
| 6 | 0.3 ± 0.0 | 1.2 ± 0.1 | 0.4 ± 0.0 | 3.1 ± 0.6 |

### Example 8

An experiment was performed to determine the effectiveness of growing Aurantiochytrium with combination of acetate and a non-acetate organic carbon sources, in this case glycerol. Aurantiochytrium sp. HS399 was grown in a 100 L open pond. The base medium contained: sodium acetate (1 g/L), glycerol (30 g/L) monosodium glutamate monohydrate (2.5 g/L) NaCl (12.5 g/L), MgSO₄.7H₂O (2.5 g/L), KCl (0.5 g/L), CaCl₂ (0.1 g/L), KH₂PO₄ (0.5 g/L), vitamins (1 ml/L), and trace metal (5 ml/L). Trace and vitamins stocks were prepared according to Ashford, et al. 2000. Lipids 35, 1377-1386. The ponds were inoculated at 1 v/v using exponentially growing cultures. Aeration was maintained at 0.5 vvm using a porous hose. In one treatment the pH was controlled at a set point of pH 7 with hydrochloric acid, and no acetic acid was supplied (i.e., glycerol only). In another treatment acetic acid was supplied by a pH auxostat system at a set point of 5.5 in addition to the glycerol. The impact on microalgae growth and contamination was analyzed by measuring dry weights (filtration), residual acetate concentration in the culture medium (HPLC), and total aerobic bacteria counts (petrifilms) daily. The results are shown in Tables 19, 20, and 21.

TABLE 19

| | Cell Dry Weight (g/L) | | Residual Acetate (g/L) | |
|---|---|---|---|---|
| | pH 5.5, | pH 7.0, | pH 5.5, | pH 7.0, |
| Time (d) | Acetic Acid | HCl | Acetic Acid | HCl |
| 0 | 0.0 ± 0.0 | 0.1 ± 0.0 | 0.5 ± 0.0 | 0.0 ± 0.0 |
| 1 | 1.1 ± 0.1 | 1.6 ± 0.0 | 1.0 ± 0.1 | 0.0 ± 0.0 |
| 2 | 3.5 ± 0.4 | 4.3 ± 0.5 | 1.4 ± 0.1 | 0.0 ± 0.0 |
| 3 | 6.2 ± 0.6 | 5.5 ± 0.9 | 1.5 ± 0.0 | 0.0 ± 0.0 |
| 4 | 7.7 ± 0.1 | 6.5 ± 1.4 | 0.9 ± 0.7 | 0.1 ± 0.0 |
| 5 | 9.7 ± 0.4 | 8.1 ± 1.2 | 1.1 ± 0.7 | 0.0 ± 0.0 |

TABLE 20

Contamination (CFU/mL)

| Time (d) | pH 5.5, Acetic Acid | pH 7.0, HCl |
|---|---|---|
| 0 | 5.5E+04 | 1.9E+05 |
| 1 | 4.8E+05 | 2.9E+10 |
| 2 | 5.7E+07 | 1.3E+16 |
| 3 | 4.0E+10 | 2.1E+18 |
| 4 | 4.6E+12 | 1.4E+17 |
| 5 | 8.0E+14 | 3.4E+21 |

TABLE 21

Glycerol (g/L)

| Time (d) | pH 5.5, Acetic Acid | pH 7.0, HCl |
|---|---|---|
| 0 | 23.0 ± 0.0 | 21.5 ± 0.7 |
| 1 | 21.0 ± 0.0 | 17.5 ± 0.7 |
| 2 | 18.5 ± 0.7 | 11.0 ± 0.0 |
| 3 | 18.0 ± 1.4 | 8.5 ± 0.7 |
| 4 | 17.5 ± 0.7 | 5.0 ± 1.4 |
| 5 | 16.0 ± 0.0 | 1.0 ± 1.4 |

As shown in Table 19, the culture receiving acetic acid had comparable growth to the other culture. As shown in Table 20, the aerobic bacteria counts decreased at least by 3 log throughout the batch for the culture receiving acetic acid as compared to the other treatment. As shown in Table 21, the consumption rate of glycerol was lower for the treatment receiving acetic acid, indicating that acetic acid is preferred over glycerol when acetate has accumulated in the cell.

### Example 9

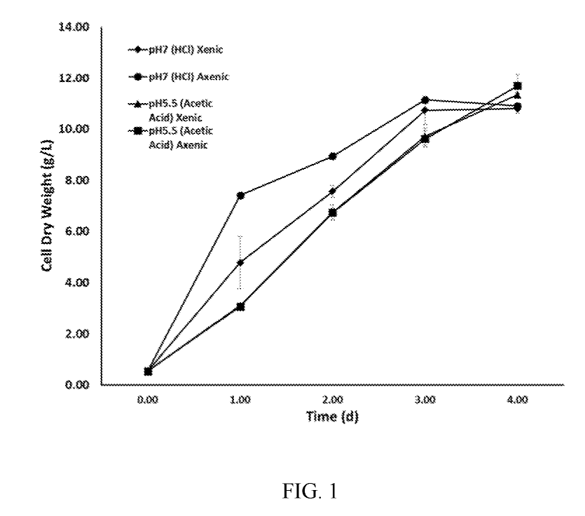

An experiment was performed to determine the ability of Aurantiochytrium to grow on combination of acetate and another organic carbon source in axenic and contaminated culture conditions. Aurantiochytrium sp. HS399 was grown in an acetic acid/pH-auxostat mode in 700 ml bubble column at 27° C. in sterile conditions. The initial culture medium was supplemented with 30 g/L glycerol and 0.5 g/L acetate. The residual acetate concentration in the culture medium was maintained at 1±0.5 g/L and treatments with a pH set point of 5.5 and 7 titrated by either acetic acid or hydrochloric acid (HCl) were compared for axenic cultures and cultures contaminated with bacteria (xenic). Cell dry weights were measured daily. Results are shown in FIG. 1. The results in FIG. 1 show that Aurantiochytrium was able to achieve comparable growth in axenic and contaminated cultures receiving acetic acid as those that did not receive acetic acid.

### Example 10

An experiment will be performed to compare the ability of multiple strains of microalgae, Aurantiochytrium sp. (HS399) and Schizochytrium limacinum (ATCC SR21). Multiple treatments for each strain will be grown in a flask with one treatment comprising 0 g/L acetate and one treatment comprising 40 g/L of acetate. The cultures will be grown at a temperature of about 27° C., agitation of about 180 rpm, and the absence of light for about 6 days. The cultures will be sampled daily to measure culture dry weight, acetate concentration, and pH.

### Example 10

An experiment will be performed to compare the effects of growing Aurantiochytrium sp. in different conditions to target the accumulation of culture cell dry weight of 100 g/L and a final percent total fatty acids of 70%. The proposed culture conditions with varied nitrogen treatments to be tested in the experiment are listed in Table 22. The control treatment will correspond to a traditional two stage fermentation process. The term “% N Inoc” corresponds to the percent nitrogen used in a the culture stage producing biomass accumulation for inoculating the final culture stage. The term “% N Batch” corresponds to the percent of nitrogen provided as a batch at the beginning of the final culture stage. The term “% N Fed” corresponds to the percentage of nitrogen provided to during the final culture stage with the organic carbon feed. The term “Inoc % Vol” corresponds to the anticipated percent solids of needed to inoculate a culture for the final culture stage.

TABLE 22

| Treatment No. | % N Inoc | % N Batch | % N Fed | Inoc % Vol |
|---|---|---|---|---|
| Control | 30 | 70 | 0 | 10 |
| 1 | 100 | 0 | 0 | 33 |
| 2 | 80 | 0 | 20 | 27 |
| 3 | 60 | 0 | 40 | 20 |
| 4 | 40 | 0 | 60 | 13 |
| 5 | 10 | 0 | 90 | 3 |
| 6 | 80 | 20 | 0 | 27 |
| 7 | 60 | 20 | 20 | 20 |
| 8 | 40 | 20 | 40 | 13 |
| 9 | 20 | 20 | 60 | 7 |
| 10 | 5 | 20 | 75 | 2 |
| 11 | 60 | 40 | 0 | 20 |
| 12 | 40 | 40 | 20 | 13 |
| 13 | 10 | 40 | 50 | 3 |
| 14 | 100 | 0 | 0 | 83 |
| 15 | 80 | 0 | 20 | 67 |
| 16 | 60 | 0 | 40 | 50 |
| 17 | 40 | 0 | 60 | 33 |
| 18 | 10 | 0 | 90 | 8 |
| 19 | 80 | 20 | 0 | 67 |
| 20 | 60 | 20 | 20 | 50 |
| 21 | 40 | 20 | 40 | 33 |
| 22 | 20 | 20 | 60 | 17 |
| 23 | 5 | 20 | 75 | 4 |
| 24 | 60 | 40 | 0 | 50 |
| 25 | 40 | 40 | 20 | 33 |
| 26 | 20 | 40 | 40 | 17 |
| 27 | 10 | 40 | 50 | 8 |
| 28 | 1 | 0 | 99 | 1 |
| 29 | 2.3 | 48.8 | 48.8 | 5 |

### ASPECTS OF THE INVENTION

In one non-limiting embodiment, a method may comprise: providing a culture of microalgae in the Thraustochytriaceae family in a culture medium suitable for biomass production: supplying an organic carbon source comprising at least one of acetate and acetic acid; and culturing the microalgae in conditions suitable to increase a biomass density of the culture.

In one non-limiting embodiment, a method may comprise: providing a culture of microalgae in the Thraustochytriaceae family in a culture medium suitable for lipid production; supplying an organic carbon source comprising at least one of acetate and acetic acid; and culturing the microalgae in conditions suitable to increase a concentration of lipids in the culture. In one aspect the increase in the concentration of lipids is any detectable amount. In another aspect, the lipid concentration is increased at least 10% as compared to the starting concentration. In further aspects, the lipid concentration is increased at least 25%, at least 50%, at least 75%, at least 100% (i.e., doubled), at least 150%, at least 200%, at least 250%, at least 300%, at least 350%, at least 400%, at least 500%, at least 600%, at least 750%, at least 900%, at least 1000%, at least 1500%6, at least 2000%, at least 2500%, at least 3000%, at least 3500%, at least 3750%, at least 4000%, or more than 4000% (such as at least 450% or at least 5000%). In some aspects the lipid concentration is increased between 100%-5000%, such as between 200%-5000%, 300%-5000%, 500%-5000%, 100%-4000%, 200%-4000%, 300%-4000%, 400%-4000%, 500%-4000%, 50%-3500%, 50%-3250%, 100%-3500%, 150%-3500%, 200%-3500%, 300%-3500%, 350%-3500%, or 500%-3500%.

In some embodiments, the supply of the organic carbon source may further comprise at least one selected from the group consisting of sucrose, fructose, molasses, xylose, glycerol, and glucose. In some embodiments, a source of nitrogen may comprise at least one of monosodium glutamate (MSG), ammonia, ammonium, urea, nitrates, and glycine.

In some embodiments, the culture may achieve a biomass weight yield per gram of organic carbon of up to 0.55. In some embodiments, the culture may achieve a biomass productivity rate of up to 60 g/L/day. In some embodiments, the culture conditions may be non-sterile. In some embodiments, the supply of at least one of acetate and acetic acid may be provided by a pH auxostat system.

In some embodiments, the culture may achieve an Omega-3 fatty acid weight yield per gram of organic carbon of up to 0.30. In some embodiments, the culture may achieve a lipid productivity rate of up to 1.0 g/L/hr. In some embodiments, the supply of at least one of acetate and acetic acid may be terminated prior to harvesting the microalgae to induce an increase in the culture pH. In some embodiments, the culture may be disposed in an open outdoor bioreactor for at least part of the culture life.

In another non-limiting embodiment, a method may comprise: providing a culture of microalgae in the Thraustochytriaceae family in an aqueous culture medium: supplying an organic carbon source comprising at least one of acetate and acetic acid; culturing the microalgae in a first set of culture conditions suitable to increase the biomass density of the culture; and culturing the microalgae in a second set of culture conditions suitable to increase a concentration of lipids in the culture.

In another non-limiting embodiment, a method may comprise: providing a culture of microalgae in the Thraustochytriaceae family in an aqueous culture medium: supplying an organic carbon source comprising at least one of acetate and acetic acid; culturing the microalgae in a first set of culture conditions suitable to increase the biomass density of the culture: and culturing the microalgae in a second set of culture conditions suitable for producing biomass accumulation through cell growth and lipid accumulation concurrently.

In some embodiments, the second set of culture conditions may comprise nitrogen sufficient conditions. In some embodiments, the second set of conditions may comprise an initial batch supply of nitrogen. In some embodiments, the second set of conditions may comprise no initial nitrogen in the aqueous culture medium. In some embodiments, the second set of conditions may comprise a supply of nitrogen fed with a supply of the organic carbon. In some embodiments, the second set of conditions occur in a non-sterilized bioreactor.

All references, including publications, patent applications, and patents, cited herein are hereby incorporated by reference in their entirety and to the same extent as if each reference were individually and specifically indicated to be incorporated by reference and were set forth in its entirety herein (to the maximum extent permitted by law), regardless of any separately provided incorporation of particular documents made elsewhere herein.

The use of the terms “a” and “an” and “the” and similar referents in the context of describing the invention are to be construed to cover both the singular and the plural, unless otherwise indicated herein or clearly contradicted by context.

Unless otherwise stated, all exact values provided herein are representative of corresponding approximate values (e.g., all exact exemplary values provided with respect to a particular factor or measurement can be considered to also provide a corresponding approximate measurement, modified by “about,” where appropriate). All provided ranges of values are intended to include the end points of the ranges, as well as values between the end points.

The description herein of any aspect or embodiment of the invention using terms such as “comprising”, “having,” “including,” or “containing” with reference to an element or elements is intended to provide support for a similar aspect or embodiment of the invention that “consists of”, “consists essentially of”, or “substantially comprises” that particular element or elements, unless otherwise stated or clearly contradicted by context (e.g., a composition described herein as comprising a particular element should be understood as also describing a composition consisting of that element, unless otherwise stated or clearly contradicted by context).

All headings and sub-headings are used herein for convenience only and should not be construed as limiting the invention in any way.

The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

The citation and incorporation of patent documents herein is done for convenience only and does not reflect any view of the validity, patentability, and/or enforceability of such patent documents.

This invention includes all modifications and equivalents of the subject matter recited in the claims and/or aspects appended hereto as permitted by applicable law.

### REFERENCES

US20150259706
US20140154755
Patil, et al. Improved synthesis of docosahexaenoic acid (DHA) using Schizochytrium limacinum SR21 and sustainable media. Chemical Engineering Journal 268 (2015) 187-196.
Xie, Y., and Wang, G. (2015). Mechanisms of fatty acid synthesis in marine fungus-like protists. Appl. Microbiol. Biotechnol. 99, 8363-8375, doi: 10.1007/s00253-015-6920-7.

1. A method, comprising:
a. Providing a culture of microalgae in the Thraustochytriaceae family in a culture medium suitable for biomass production;
b. Supplying an organic carbon source comprising at least one of acetate and acetic acid; and
c. Culturing the microalgae in conditions suitable to increase a biomass density of the culture by at least 200%.

2. The method of claim 1, wherein the supply of the organic carbon source further comprises at least one selected from the group consisting of sucrose, fructose, molasses, xylose, glycerol and glucose.

3. The method of claim 1, further comprising the step of supplying a source of nitrogen, wherein the source of nitrogen comprises at least one of monosodium glutamate (MSG), ammonia, ammonium, urea, nitrates, and glycine.

4. The method of claim 1, wherein the culture achieves a biomass weight yield per gram of organic carbon of up to 0.55.

5. The method of claim 1, wherein the culture achieves a biomass productivity rate of up to 60 g/L/day.

6. The method of claim 1, wherein the culture conditions are non-sterile.

7. The method of claim 1, wherein the supply of at least one of acetate and acetic acid is provided by a pH auxostat system.

8. A method, comprising:
a. Providing a culture of microalgae in the Thraustochytriaceae family in a culture medium suitable for lipid production;
b. Supplying an organic carbon source comprising at least one of acetate and acetic acid; and
c. Culturing the microalgae in conditions suitable to increase a concentration of lipids in the culture by at least 200%.

9. The method of claim 8, wherein the supply of the organic carbon source further comprises at least one of sucrose, fructose, molasses, xylose, glycerol and glucose.

10. The method of claim 8, further comprising the step of supplying a source of nitrogen, wherein the source of nitrogen comprises at least one of monosodium glutamate (MSG), ammonia, ammonium, urea, nitrates, and glycine.

11. The method of claim 8, wherein the culture achieves an Omega-3 fatty acid weight yield per gram of organic carbon of up to 0.30.

12. The method of claim 8, wherein the culture achieves a lipid productivity rate of up to 1.0 g/L/hr.

13. The method of claim 8, wherein the culture conditions are non-axenic.

14. The method of claim 8, wherein the supply of at least one of acetate and acetic acid is provided by a pH auxostat system.

15. The method of claim 8, wherein the supply of at least one of acetate and acetic acid is terminated prior to harvesting the microalgae to induce an increase in culture pH.

16. A method, comprising:
a. Providing a culture of microalgae in the Thraustochytriaceae family in an aqueous culture medium;
b. Supplying an organic carbon source comprising at least one of acetate and acetic acid;
c. Culturing the microalgae in a first set of culture conditions suitable to increase a biomass density of the culture; and
d. Culturing the microalgae in second set of culture conditions suitable to increase a concentration of lipids in the culture.

17. The method of claim 16, wherein the culture is disposed in an open bioreactor for at least part of the culture life.

18. A method, comprising:
a. Providing a culture of microalgae in the Thraustochytriaceae family in an aqueous culture medium;
b. Supplying an organic carbon source comprising at least one of acetate and acetic acid;
c. Culturing the microalgae in a first set of culture conditions suitable to increase a biomass density of the culture; and
d. Culturing the microalgae in a second set of culture conditions suitable for producing biomass accumulation through cell growth and lipid accumulation concurrently.

19. The method of claim 18, wherein the second set of culture conditions comprise nitrogen sufficient conditions.

20. The method of claim 18, wherein the second set of conditions comprise an initial batch supply of nitrogen.

21. The method of claim 18, wherein the second set of conditions comprise no initial nitrogen in the aqueous culture medium.

22. The method of claim 18, wherein the second set of conditions comprise a supply of nitrogen fed with a supply of the organic carbon.

23. The method of claim 18, wherein the second set of conditions occur in a non-sterilized bioreactor.
